(19) United States
(12) Patent Application Publication
(10) Pub. No.: US 2011/0105797 A1
(43) Pub. Date: May 5, 2011

(54) Creatinol sulfate and synthesis method thereof

(75) Inventors: Guoji Zhang, Tianjin (CN); Jiashu Zhang, Diamond Bar, CA (US)

(73) Assignee: Tianjin Tiancheng Pharmaceutical Co., Ltd.

(21) Appl. No.: 12/590,139

(22) Filed: Nov. 2, 2009

(51) Int. Cl. C07C 277/02

(52) U.S. Cl. 564/241

(57) ABSTRACT

A chemical compound of creatinol sulfate is formed by chemically reacting N-methyl-amino-ethanol, cyanamide, with sulfuric acid to produce the creatinol sulfate with relatively higher yield rate and purity, wherein the mol ratio of N-methyl-amino-ethanol, sulfuric acid, and cyanamide is approximately 2:1:2.

### BACKGROUND OF THE PRESENT INVENTION

1. Field of Invention

The present invention relates to the chemical compounds of creatinol sulfate, and more particularly to a method for synthesizing the creatinol sulfate.

2. Description of Related Arts

There are lots of chemical compounds are developed for increasing the durability of human muscle. For example, the creatine phosphate is well known for storing energy in muscles, prolonging the durability of muscle, and delaying the muscle fatigue occurring. The creatine phosphate is strongly involved into the process of synthesizing ATP in muscles, which is playing the role of transporting chemical energy within cells for metabolism. When a human being, such as an athlete, is doing exercise, the ATP in muscles is gradually reducing to converting and providing energy to the muscles. The creatine phosphate is able to convert ADP in muscles into ATP, so that the muscle is able to recover relatively sooner.

Generally, the creatine phosphate converts the ADP in muscles to ATP by donating the phosphate group of the creatine phosphate, so that during the intense activity of the human body, the creatine phosphate plays an important role for quickly regenerating the ATP from ADP, so as to reserve the energy of the muscle.

Accordingly, the present invention preferably uses the creatinol sulfate instead of the higher price of the S-ethyl isothiourea hydrobromide, so as to produce relatively less toxicity and reduce the damages of the environments and human bodies.

The creatinol sulfate is one of the important intermediums of organic synthesis. The creatinol sulfate may also be used as an energy supplements, food additives and plant growth regulator.

Traditionally, the creatinol sulfate is synthesized via the S-methyl-isothiourea sulfate chemically reacting with N-methyl-amino-ethanol in water solution, and then adding a large amount of acetone to precipitate the creatinol sulfate crystals. However, it is a waste that the great amount of acetone of organic solvent is needed to be added in the synthesis process. While the synthesizing of the creatinol sulfate, the toxic gaseous of methanethiol is generated to damage the health of the operators and the environment.

The present invention uses N-methyl amino ethanol and 30% cyanamide as the raw material for synthesizing the creatinol sulfate. The entirely reactions of synthesis are under the temperature of 10 to 90 for 1 to 5 hours, so that the no complicated operations or critical conditions are required during the synthesis process, so as to save the energy. The present invention also uses the water to partially replace the organic mixture, so as to reduce the volatile amount of the organic solvent. Therefore, the “after treatment” of the waste of the side products produced during the synthesis process is relatively easier. The manufacturing cost for synthesis is minimized. The higher purity of the synthesized creatinol sulfate is obtained after the synthesizing process of the present invention.

### SUMMARY OF THE PRESENT INVENTION

An object of the present invention is to provide a method for synthesizing chemical compound of creatinol sulfate.

Another object of the present invention is to provide a method for synthesizing creatinol sulfate, wherein the yield rate and purity of the creatinol sulfate is relatively higher.

Another object of the present invention is to provide a method for synthesizing creatinol sulfate, wherein the creatinol sulfate is formed by N-methyl-amino-ethanol chemically reacting with cyanamide solution.

Another object of the present invention is to provide a method for synthesizing creatinol sulfate, wherein the reaction temperature for forming the creatinol sulfate is around 30° C. to 70° C. for 1 to 5 hours, so as to minimize the consuming of energy.

Another object of the present invention is to provide a method for synthesizing creatinol sulfate, wherein water is mainly used as the solvent during the reaction of forming the creatinol, so as to minimize the toxicity and simplify the waste treatment process after the reaction is finished.

Another object of the present invention is to provide a cyanamide reacting with a methanol and a sulfuric acid to react with N-methyl-amino-ethanol to form a sulfate compound, so as to form the creatinol sulfate.

Accordingly, in order to accomplish the above objects, the present invention provides a chemical compound of creatinol sulfate, comprising the following chemical structure:

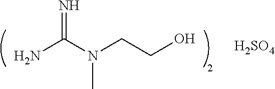

wherein said creatinol sulfate is formed by N-methyl-amino-ethanol chemically reacted with cyanamide.

The present invention further provides a synthesizing process of creatinol sulfate, comprising the steps of:

(a) mixing a predetermined amount of N-methyl-amino-ethanol with a predetermined amount of methanol to form a first mixture;

(b) adding a predetermined amount of sulfuric acid into the first mixture to form a second mixture;

(c) add a predetermined amount of cyanamide into the second mixture to form a third mixture;

(d) add a predetermined amount of ethanol into the third mixture to precipitate out creatinol sulfate;

(e) dry said precipitated creatinol sulfate to obtain a final product of creatinol sulfate having 60 to 80% of yield.

These and other objectives, features, and advantages of the present invention will become apparent from the following detailed description, the accompanying drawings, and the appended claims.

### BRIEF DESCRIPTION OF THE DRAWINGS

FIG. 1 is a flow chart of a process of synthesizing creatinol sulfate according to a preferred embodiment of the present invention.

### DETAILED DESCRIPTION OF THE PREFERRED EMBODIMENT

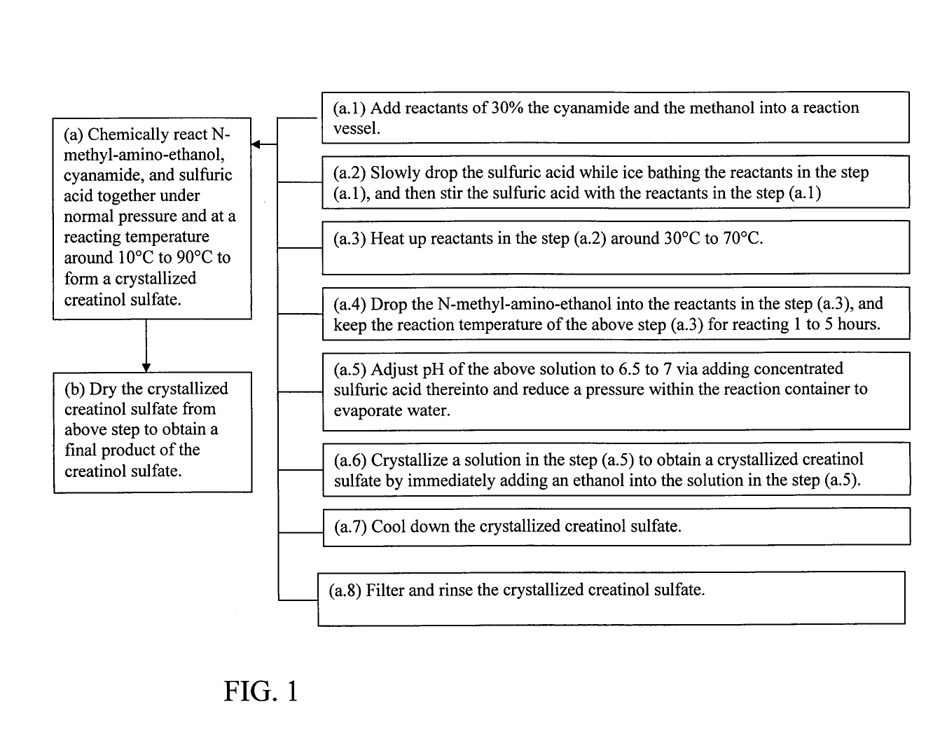

Referring to FIG. 1 of the drawing, a process for synthesizing an organic compound of creatinol sulfate is illustrated. The creatinol sulfate, also named 1-methyl-1-β-(hydroxy ethyl) guanidine sulfate, has a molecular formula of C₈H₂₄N₆O₆S, molecular weight of 332, and a CAS registration number of 50648-53-8. The creatinol sulfate, which also represents N-(2-Hydroxyethyl)-N-methylguanidine sulfate, has the following chemical structure:

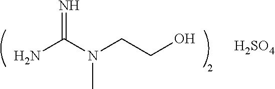

Accordingly, the preferred embodiment, the creatinol sulfate is preferably formed from N-methyl-amino-ethanol chemically reacting with cyanamide and concentrated sulfuric acid to form the creatinol sulfate.

As shown in FIG. 1, a synthesis process for producing the creatinol sulfate generally comprises the following steps.

(1) Mix a predetermined amount of N-methyl-amino-ethanol with a predetermined amount of methanol to form a first mixture.

(2) Add a predetermined amount of sulfuric acid into the first mixture to form a second mixture.

(3) Add a predetermined amount of cyanamide into the second mixture to form a third mixture.

(4) Add a predetermined amount of ethanol into the third mixture to form a crystallized creatinol sulfate.

(5) Dry the crystallized creatinol sulfate to obtain a final product of creatinol sulfate.

Accordingly, a 250 ml four-necked, round bottomed flask is preferably used as the reaction container to mix N-methyl-amino-ethanol with the methanol and to contain the first mixture in the step (1). In addition, 37.5 g (0.5 mol) of N-methyl-amino-ethanol and 20 ml of methanol are respectively used in ratio to form the first mixture in the step (1).

Before the step (2), i.e. before adding the sulfuric acid into the first mixture, the first mixture is preferably cooled down at a position below 10° C.

According to the step (2), the sulfuric acid is added into the first mixture by slowing dropping the sulfuric acid into the first mixture. Preferably, 14 ml (0.26 mol) of concentrated sulfuric acid is used in the step (2).

The step (2) further comprises the step of:

(2.1) stirring the second mixture preferably at room temperature and for 0.5-1 hour; and

(2.2) heating up the second mixture between 30° C. and 70° C., preferably 50° C., before the cyanamide is added thereto.

In the step (3), the cyanamide is slowly added into the second mixture preferably by dropping the cyanamide thereinto. Preferably, 30% (80.5 ml, 0.575 mol) of cyanamide aqueous is added into the second mixture, wherein the third mixture, i.e. the second mixture with cyanamide, is kept at the reaction temperature for 1 to 5 hours of reaction time. Accordingly, the mol ratio of N-methyl-amino-ethanol, sulfuric acid, and cyanamide is preferably 2:1:2.

After the step (3), the present invention further comprises the following steps.

(3.1) Adjust pH value of the third mixture between 6.5 and 7 by adding concentrated sulfuric acid thereinto.

(3.2) Reduce a pressure and evaporate water of the third mixture within the reaction vessel.

According to the step (4), 100 ml of ethanol is preferably added into the third mixture when the third mixture is still warm. In other words, the third mixture is kept above the room temperature, between 30° C. and 70° C., when the ethanol is added to precipitate out creatinol sulfate.

After the ethanol is added, the step (4) further comprises the following steps.

(4.1) Cool down the third mixture at a temperature below 20° C.

(4.2) Remove the crystallized creatinol sulfate from the third mixture preferably by filtration.

(4.3) Rinse the crystallized creatinol sulfate by additional ethanol.

Accordingly, the precipitated creatinol sulfate is filtered and rinsed. A vacuum filtration method is preferably provided for filtering the crystallized creatinol sulfate, and ethanol is further provided for rinsing the crystallized creatinol sulfate for removing undesired side products and increasing the drying rate.

Therefore, 60 to 80% yield rate of the final product of creatinol sulfate (65 g, 78% yield) is obtained after the creatinol sulfate is dried in the step (5).

Accordingly, the overall reaction of the creatinol sulfate forming by chemically reacting N-methyl-amino-ethanol with cyanamide and sulfuric acid is shown below.

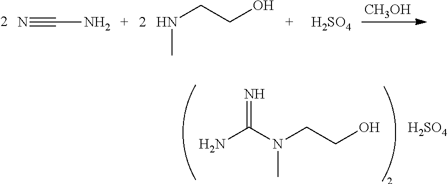

According to the preferred embodiment, creatinol sulfate of the present invention can be used as a chemical compound to form creatinol O-phosphate by chemically reacting creational sulfate with a chemical compound selected from the group consisting of POCl₃, ClPO₃OH₂, and P₂O₅.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A chemical compound of creatinol sulfate, comprising the following chemical structure: 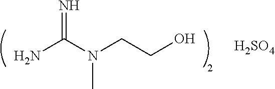
wherein said creatinol sulfate is formed by N-methyl-amino-ethanol chemically reacted with cyanamide.

2. The chemical compound of creatinol sulfate, as recited in claim 1, wherein sulfuric acid is mixed with said N-methyl-amino-ethanol before said N-methyl-amino-ethanol chemically reacts with said cyanamide to form said creatinol sulfate.

3. The chemical compound of creatinol sulfate, as recited in claim 2, wherein a predetermined amount of methanol is mixed with said N-methyl-amino-ethanol before said N-methyl-amino-ethanol is chemically reacted with said sulfuric acid.

4. The chemical compound of creatinol sulfate, as recited in claim 3, wherein a predetermined amount of ethanol is added into said N-methyl-amino-ethanol, said sulfuric acid, and said cyanamide to crystallize said creatinol sulfate.

5. The chemical compound of creatinol sulfate, as recited in claim 4, wherein the mol ratio of said N-methyl-amino-ethanol, said sulfuric acid, and said cyanamide is approximately 2:1:2.

6. A synthesizing process of creatinol sulfate, comprising the steps of:
(a) mixing a predetermined amount of N-methyl-amino-ethanol with a predetermined amount of methanol to form a first mixture;
(b) adding a predetermined amount of sulfuric acid into said first mixture to form a second mixture;
(c) adding a predetermined amount of cyanamide into said second mixture to form a third mixture;
(d) adding a predetermined amount of ethanol into said third mixture to form a crystallized creatinol sulfate;
(e) drying said crystallized creatinol sulfate to obtain a final product of creatinol sulfate having 60 to 80% of yield rate.

7. The method, as recited in claim 6, wherein the mol ratio of said N-methyl-amino-ethanol, said sulfuric acid, and said cyanamide is approximately 2:1:2.

8. The method as recited in claim 6 wherein, in the step (b), said sulfuric acid is added by slowing dropping said sulfuric acid into said first mixture.

9. The method as recited in claim 7 wherein, in the step (b), said sulfuric acid is added by slowing dropping said sulfuric acid into said first mixture.

10. The method as recited in claim 9 wherein, before the step (b), said first mixture is cooled down at a temperature below 10° C.

11. The method, as recited in claim 10, wherein the step (b) further comprises the steps of:
(b.1) stirring said second mixture at room temperature and for 0.5-1 hour; and
(b.2) heating up said second mixture between 30° C. and 70° C. before said cyanamide is added thereto.

12. The method, as recited in claim 6, wherein the step (c) further comprises the steps of:
(c.1) adjusting pH value of said third mixture between 6.5 to 7; and
(c.2) evaporating water from said third mixture.

13. The method, as recited in claim 11, wherein the step (c) further comprises the steps of:
(c.1) adjusting pH value of said third mixture between 6.5 to 7; and
(c.2) evaporating water from said third mixture.

14. The method, as recited in claim 12, wherein, in the step (c.1), the pH value of said third mixture is adjusted by additional sulfuric acid.

15. The method, as recited in claim 13, wherein, in the step (c.1), the pH value of said third mixture is adjusted by additional sulfuric acid.

16. The method, as recited in claim 6, wherein the step (d) further comprises the steps of:
(d.1) cooling down said third mixture at a temperature below 20° C.;
(d.2) removing said crystallized creatinol sulfate from said third mixture; and
(d.3) rinsing said crystallized creatinol sulfate.

17. The method, as recited in claim 15, wherein the step (d) further comprises the steps of:
(d.1) cooling down said third mixture at a temperature below 20° C.;
(d.2) removing said crystallized creatinol sulfate from said third mixture; and
(d.3) rinsing said crystallized creatinol sulfate.

18. The method, as recited in claim 16, wherein, in the step (d.3), said crystallized creatinol sulfate is rinsed by additional ethanol.

19. The method, as recited in claim 17, wherein, in the step (d.3), said crystallized creatinol sulfate is rinsed by additional ethanol.

20. The method, as recited in claim 6, wherein, in the step (d), said ethanol into said third mixture to precipitate out said creatinol sulfate when said third mixture is warm.

21. The method, as recited in claim 19, wherein, in the step (d), said ethanol into said third mixture to precipitate out said creatinol sulfate when said third mixture is warm.
